(19) United States
(12) Patent Application Publication
(10) Pub. No.: US 2004/0126813 A1
(43) Pub. Date: Jul. 1, 2004

(54) Systems and methods for sorting protein sequences and structures for visualization

(76) Inventors: Derek A. Debe, Sierra Madre, CA (US); Jeffrey Regan, Dubuque, IA (US); Joseph F. Danzer, Pasadena, CA (US)

Correspondence Address: PERKINS COIE LLP, POST OFFICE BOX 1208, SEATTLE, WA, 98111-1208, US

(21) Appl. No.: 10/652,866

(22) Filed: Aug. 29, 2003

Related U.S. Application Data
(60) Provisional application No. 60/406,870, filed on Aug. 29, 2002.

(51) Int. Cl. G01N 33/53; G06F 19/00; G01N 33/48; G01N 33/50

(52) U.S. Cl. 435/7.1; 702/19

(57) ABSTRACT

The present invention relates to methods and systems for sorting a plurality of protein structures generated by a database search based upon their corresponding sequences. The methods and systems according to the invention are based upon computer generated graphical user interfaces that present to the user: i) a multiple sequence alignment of the sequences corresponding to the structures to be sorted; ii) the means to identify whether one or more of the sequences comprise homologous alignment domains; and iii) the means to select one or more sequences for subsequent processing of their corresponding structures. In another aspect of the invention, the user is presented one or more phylogenetic tree representations of user selected homologous alignment domains so that the user may quickly determine the evolutionary similarity of a family of homologous alignment domains.

### CROSS-REFERENCE TO RELATED APPLICATIONS

This application claims priority to U.S. Provisional Patent Application Serial No. 60/406,870, filed Aug. 29, 2002, the disclosure of which is incorporated by reference herein in its entirety.

### FIELD OF THE INVENTION

The present invention relates to a computer system programmed for manipulating and visualizing a plurality of protein structures and protein sequences.

### BACKGROUND

Guided by large public and private databases first developed for genomes and now for proteomes, molecular biology is transitioning from the laboratory bench to the computer desktop. Computational biologists mine large databases using homology search tools and functional annotation tools to identify and characterize putative drug targets.

Current efforts to identify protein based drug targets focus on a small number of “drug-proven” protein families such as kinases, proteases, nuclear hormone receptors, transmembrane proteins, chemokines and cytokines. These protein families are referred to as “drug-proven” because a number of proven drugs and validated screened targets are based upon proteins found in these families. In order to maximize the number of new drugs brought to market (currently, only about 5% of drug development projects reach the market) many companies are directing their efforts on identifying and characterizing novel members of drug-proven protein families through genome-wide sequence homology searching.

Assume a computational biologist knows the sequence and/or structure of a particular proven drug target and, in order to identify new putative drug targets, wishes to determine all the known sequences with at least 60% homology to the know drug target's sequence and their corresponding structures. To this end, the computational biologist may query a protein sequence database, such as the protein data bank (“PDB”) for all the sequences which are at least 60% homologous to the sequence of the known target using a primary sequence comparison tool such as one of the BLAST, Smith-Waterman, Hidden Markov Model (“HMM”) or FASTA algorithms. Until recently, most such database searches were limited to sequence comparisons. Structure based searching was far rarer because until recently, only a very small fraction of the known protein sequences could be assigned three dimensional structures. But with the advent of sophisticated homology modeling methods, such as Eidogen Corp.'s (Pasadena, Calif.), STRUCTFAST algorithm, and the development of large homology modeled protein structure databases, such as Eidogen Corp.'s Target Informatics Platform, this is no longer the case. Now, large homology modeled structure databases may be queried based upon sequence or structure with a query returning both protein sequences and their corresponding structures. Since it is not practical to simultaneously view dozens of structures at once, nor is it practical to “page through” dozens of structures to determine their relevant biological similarities/dissimilarities, there is a need for a system which allows the user to understand the relevant biological relationships between a plurality of structures before the structures are viewed. Accordingly, the present invention is directed to systems and methods for sorting a plurality of structures and sequences for subsequent processing or visualization.

### BRIEF DESCRIPTION OF THE FIGURES

FIG. 1 illustrates one method according to the invention for sorting a plurality of protein structures generated from a database query based upon their corresponding sequences.

FIG. 2 illustrates a graphical user interface used by the methods according to the invention.

FIG. 3 illustrates another graphical user interface used by the methods according to the invention.

FIG. 4 illustrates an alternative graphical user interface that may be used by the methods according to the invention.

FIG. 5 illustrates another method according to the invention for sorting a plurality of protein structures generated from a database query based upon their corresponding sequences.

FIGS. 6a-b illustrate two graphical user interfaces that may be used by the method illustrated in FIG. 5.

FIGS. 7a-c illustrate an exemplary evolutionary distance matrix and its corresponding phylogenetic tree representations that may be used by the methods according to the invention.

FIG. 8 illustrates another method according to the invention for sorting a plurality of protein structures generated from a database query based upon their corresponding sequences.

FIG. 9 illustrates another graphical user interface that may be used by the methods illustrated in FIG. 8.

FIGS. 10a-b illustrate two graphical user interfaces that may be used by the methods illustrated in FIG. 8.

FIG. 11 illustrates a system according to the invention.

### SUMMARY OF THE INVENTION

The methods and systems according to the invention relate to sorting a plurality of protein sequences and their corresponding structures generated by a search of a large protein structure/sequence database. The systems according to the invention may be based upon network servers or desktop personal computers comprising programming for the protein sorting methods according to the invention. The protein sorting methods according to the invention present to the user one or more graphical user interfaces (“GUIs”) which a user manipulates in order to understand sequence, structure and function similarities between the basket of proteins and their corresponding sequences. One method according to the invention for sorting a plurality of sequences and their corresponding structures comprises the steps of: 1) identifying a master sequence among the sequences returned from the database search; 2) displaying to the user a GUI comprising: i) a multiple sequence alignment between the master sequence and the other sequences, ii) a means to select one or more alignment domains in the master sequence; iii) a means to select a new master sequence; and iv) a means to select one or more sequences for subsequent processing of their corresponding structures; 3) receiving one or more master sequence alignment domain selections made by a user; 4) displaying to the user a second GUI comprising: i) a second multiple sequence alignment representation wherein the sequences that comprise the multiple sequence alignment are shifted such that the selected master sequence alignment domain(s) are aligned with their respective homologous alignment domains that comprise the other sequences in the multiple sequence alignment and ii) for each selected master sequence domain, a phylogenetic tree representation of a selected master sequence alignment domain and its homologous domains found in the other sequences; and 5) receiving one or more sequence selections made by the user thereby selecting their corresponding structures for subsequent processing. Another method according to the invention further displays to the user a third GUI comprising a table consisting of data entry fields that indicate the source and name of each sequence that comprises the multiple sequence alignment representation.

### DETAILED DESCRIPTION OF THE INVENTION

Methods According to the Invention

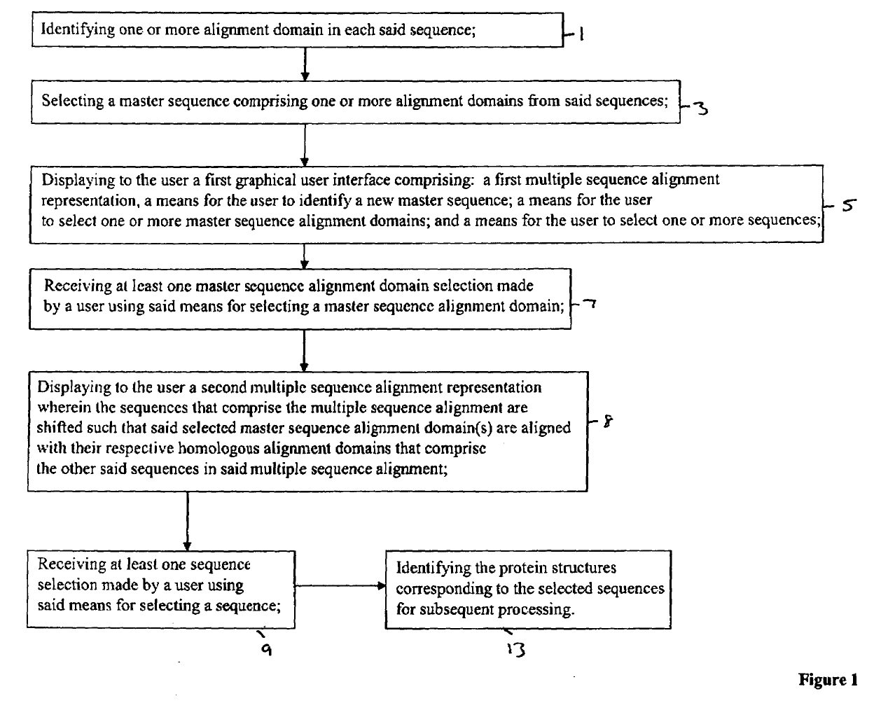

FIG. 1 illustrates one method according to the invention for sorting a plurality of protein structures. Although FIG. 1 teaches a method according to the invention in the context of sorting a plurality of protein structures generated from a database query, the methods according to the invention may be equally applied to sorting a plurality of sequences without regard to their corresponding structures. As used herein a protein structure refers to the three dimensional representation of a peptide or a protein. As used herein, a sequence corresponding to a protein structure refers to the primary sequence corresponding to a protein structure. The methods according to the invention relate to computer generated graphical user interfaces that allow a user to rapidly parse a basket of protein structures based upon the presence of annotated sequence domains (or structure domains) and the evolutionary relationships between these domains. Accordingly, it will be appreciated that protein structures are represented within a computer in the form of coordinate structure files, and sequences are represented in the form of residue string files. The graphical user interfaces (“GUIs”), and various means for interacting with graphical user interfaces, such as, cursors, menu bars, pull down menus, dialog boxes, radio boxes, check boxes and selectable objects, used by the methods according to the invention, may be implemented in any current or future programming language used for developing GUIs. Exemplary, suitable, current languages include, but are not limited to, Java, HTML, C++, C, Flash and Shockwave. As used herein, a selectable object refers to an object displayed to a user that may be selected by the user for an action by moving a cursor over the object with an input device and activating it with one or more input commands. Exemplary forms of selecting a selectable object include, but are not limited to, clicking on it, double clicking on it, right or center clicking on it with a three button input device, dragging a cursor over it or any combination thereof.

A first step 1 in the method illustrated in FIG. 1, identifies one or more alignment domains in each sequence corresponding to a structure returned from a database query. An alignment domain refers to: 1) to a sequence domain that is evolutionarily conserved across related sequences, or 2) the sequence domain corresponding to a structural fold that is conserved across related structures. One method for annotating one more alignment domains in a sequence uses RPS-BLAST in combination with a database of annotated sequence domains or sequence profiles such as the Pfam Database, the SMART Database, or the COG database to search a sequence for domains annotated in these respective databases. Links to each of these databases and a link for downloading RPS-BLAST is available at http://www.ncbi.nlm.nih.gov/Structure/cdd/cdd.shtml. Cut-off e-values for identifying a sequence domain may vary depending upon the required confidence of a domain identification, but an exemplary, suitable, cut-off e-value ranges from 10⁻⁴ to 10⁻², with 10⁻³ preferred.

Alignment domains may also be based upon structure-structure alignment domains. If a sequence corresponding to a structure returned from a database query is in the Protein Data Bank, and annotated in the SCOP database, http://scop.berkeley.edu/, SCOP structural annotations may be used to annotate it. In addition to using domain databases, one or more sequence domains may be identified in a sequence (referred to as the query sequence) by a method comprising the steps of: 1) determining a plurality of related template sequences to the query sequence using a sequence alignment tool such as the various BLAST, Smith-Waterman or FASTA algorithms and a large protein sequence database such as the NCBI Protein Sequence Database, http://www.ncbi.nlm.nih.gov/; 2) identifying putative conserved domains in the template sequences based upon their alignments with the query sequence; 3) performing a multiple sequence alignment on the related template sequences using a multiple sequence alignment tool such as ClustalW, http://www.ebi.ac.uk/clustalw/; 4) identifying one or more conserved domains from the multiple sequence alignment; 5) determining one or more domain profiles or Hidden Markov Models (“HMMs”) of the domains identified in step 4); and 6) identifying an alignment domain with a profile or HMM determined in step 5). A domain profile may be determined from a domain identified in a multiple sequence alignment using PSI-BLAST, http://www4.ncbi.nlm.nih.gov/Education/BLASTinfo/psi1.html. A Hidden Markov Model may be built from a multiple sequence alignment using HMMER, available for download at http://hmmer.wustl.edu/. Methods for determining putative conserved alignment domains based upon alignment data are within the capacity of one ordinarily skilled in the art. Sonnhammer, E. L. L., and Kahn, D., Modular Arrangement of Proteins as Inferredfrom Analysis of Homology, PROTEINS: Structure, Function and Genetics, 3:482-492 (1994) discusses the methods for identifying one or more putative conserved alignment domains from sequence alignment data. Sonnhammer, E. L. L., Eddy, S. R., and Durbin, R., Pfam: A Comprehensive Database of Protein Domain Familes Based on Seed Alignments, PROTEINS: Structure, Function and Genetics, 28:405-420 (1997) details how the alignment domains in the PFAM database are determined.

A second step 3, determines a default master sequence. A master sequence refers to a user selected sequence that controls how domains and sequences are displayed to the user in the multiple sequence alignment representation. The alignment domains that comprise a master sequence are referred to as master sequence alignment domains. There is no inherent limitation on how the default master sequence may be identified among the sequences corresponding to the structures returned from a database query. One convenient method identifies the default master sequence with the sequence comprising the greatest number of alignment domains. Another scheme identifies the master sequence with the sequence that is evolutionarily closest to the sequence which was the query sequence to the database search.

A third step 5, displays to the user a graphical user interface comprising: 1) a multiple sequence alignment representation of the sequences corresponding to the structures returned from a database query; 2) a means for the user to identify a new master sequence; 3) a means for a user to select one or more alignment domains in the master sequence; and 4) a means for the user to select one or mores sequences for subsequent processing of their corresponding structures. A multiple sequence alignment representation as used herein refers to a visual representation comprising: 1) a multiple sequence alignment between the master sequence and the remaining sequences corresponding to the structures returned from a database query; 2) identifications of a plurality of master sequence alignment domains and 3) identifications of a plurality of alignment domains that are homologous to master sequence alignment domains in the other sequences that comprise the multiple sequence alignment. A multiple sequence alignment may be formed by reference to the start and stop points of the alignment domains that comprise its constituent sequences. While the usefulness of the methods according to the invention increases as the number of alignment domains are identified in the master sequence and its related sequences, it is not necessary to identify each alignment domain in every sequence. Accordingly, a plurality of alignment domains may be sufficiently identified rather than identifying each alignment domain.

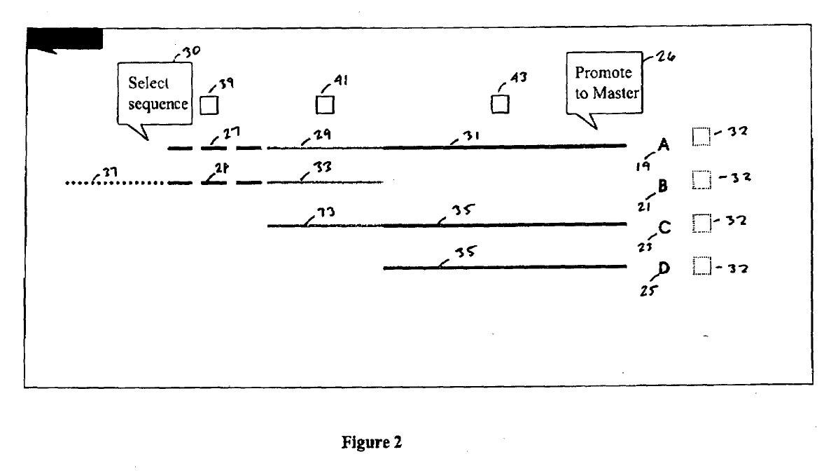

FIG. 2 illustrates, for the case of four sequences 19, 21, 23, 25, an exemplary GUI comprising: 1) a multiple sequence alignment representation of the sequences corresponding to the structures returned from a database query; 2) a means for the user to identify a new master sequence; 3) a means for a user to select one or more alignment domains in the master sequence; and 4) a means for the user to select one or more sequences for subsequent processing of their corresponding structures. The uppermost sequence is the default master sequence 19. It comprises three master sequence alignment domains, 27, 29, 31. Since it comprises three master sequence alignment domains 27, 29, 31, the user is also presented three corresponding check boxes 39, 41, 43 as a means for selecting a master sequence alignment domain(s). In this embodiment, a sequence is selected as a master sequence by moving a cursor over a sequence with an input device and selecting it via a second user activated dialog box 26. Similarly, a sequence is selected for the purposes of selecting its corresponding structure by moving a cursor over a sequence with an input device and selecting it via a second user activated dialog box 30. Alternatively, the user could be presented a second set of check of boxes 32 or even a pull-down menu 34. Homologous domains to master sequence alignment domains are identified by their corresponding graphical representations. For example, the alignment domains 33 in sequences B 21 and C 23 indicate that sequences B 21 and C 23 comprise homologous alignment domains to the second master sequence alignment domain 29. Similarly, sequence B 21 further comprises an alignment domain 28 homologous to the second master sequence alignment domain 29 and sequences C 23 and D 25 comprise homologous alignment domains 35 to the third master sequence alignment domain 31. Alignment domain 37 is not related to any other alignment domains.

Instead of check boxes, each alignment domain may be selected by either clicking on it with a cursor and an input device, or by dragging a cursor over the domain with an input device. In one embodiment of the invention, each alignment domain is represented by a different color. Alternatively, different alignment domains may be represented by varying gray scales, line thicknesses or even lines formed by repeating sub-units of points, dashed or a combination thereof. There is no inherent limitation on how alignment domains may be represented. Still further variations on this GUI may include a representation of the consensus sequence corresponding to the multiple sequence alignment and/or a histogram indicating the residue conservation down a column in the multiple sequence alignment.

A fourth step 7, receives one or more master sequence alignment domain selections made by a user using the means for selecting a master sequence alignment domain.

A fifth step 8, displays to the user a second, revised multiple sequence alignment representation where the sequence that comprise the multiple sequence alignment are shifted in order to align master sequence alignment domains selected by the user in step 4) with their homologous alignment domains in the other sequences.

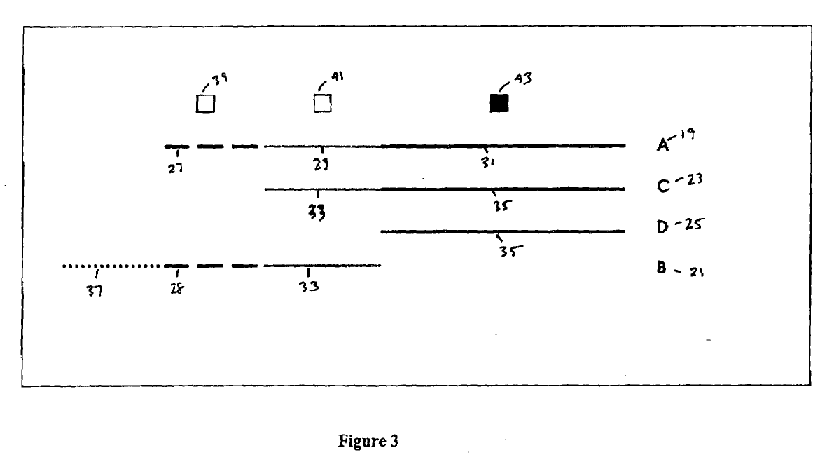

FIG. 3 illustrates the GUI illustrated in FIG. 2 after a user has selected the third check box 43 corresponding to the third master sequence alignment domain 31. By selecting the third check box 43, the sequences are arranged vertically such that the two sequences, C 23 and D 25, which each comprise an alignment domain 35 homologous to the third master sequence alignment domain 31 are grouped together, and shifted horizontally about this domain 31.

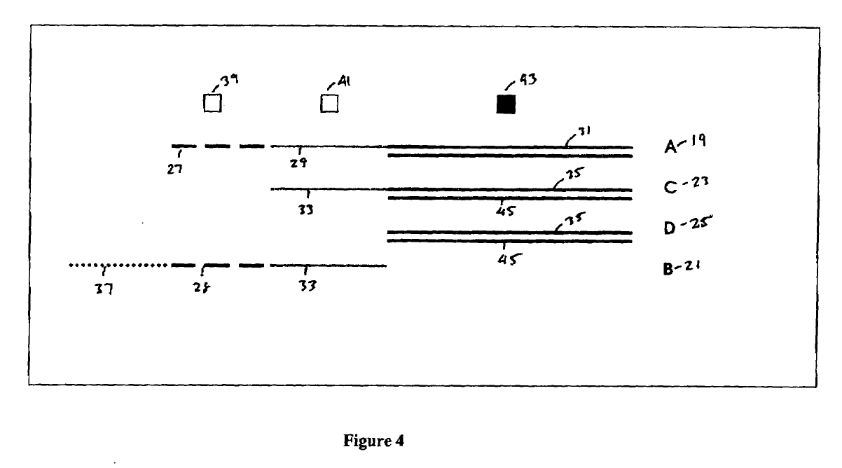

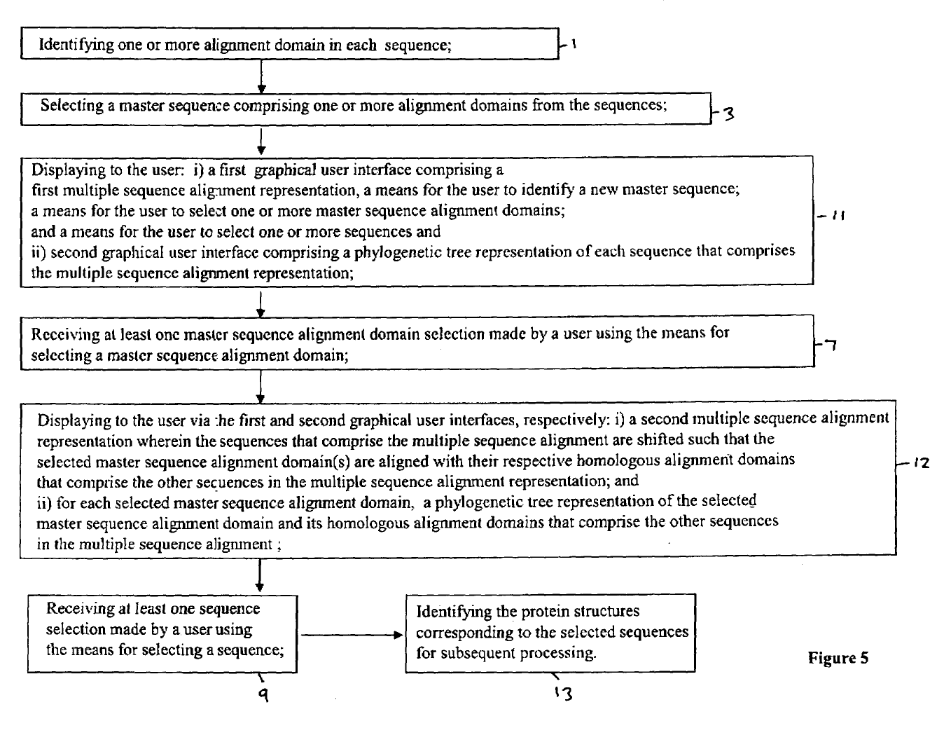

FIG. 4 illustrates an alternative multiple sequence alignment representation where annotated domains corresponding to a particular domain database, for example, the Pfam database, are indicated by a second line 45.

A sixth step 9, receives one or more sequence selections made by the user using the means presented to the user for selecting a sequence thereby selecting 13 their corresponding structures for subsequent processing. Protein structure processing refers to the processing of the structure coordinate files corresponding to the selected proteins. Exemplary processing includes but is not limited to, visualization of the protein structure or further proteomic analysis such as, fold identification, the identification of functional sites on the protein, virtual ligand screening or small molecule docking. As one ordinarily skilled in the art will appreciate, the methods according to the invention are agnostic as to the nature of the post selection protein structure processing. If a structure has been selected for visualization, its coordinate file may be processed with protein structure viewing software in order to display a three dimensional model of the protein to a user. Exemplary software for displaying protein structures includes but is not limited to, Rasmol, available for download at http://www.rasmol.org/, Cn3D available for download at http://www.ncbi.nlm.nih.gov/Structure/CN3D/cn3d.shtml, Molscript, available for download at, http://www.avatar.se/molscript/, MolMol available for download at http://www.mol.biol.ethz.ch/wuthrich/software/molmol/, and the Insight II software suite available from Accelrys, Inc., (San Diego, Calif.). For those structure viewers that recognize PDB structure files, a protein structure file may be formatted accordingly. See http://www.rcsb.org/pdb/docs/format/pdbguide2.2/guide2.2_frame.html. For those current or future viewers that do not recognize PDB structure files, a script may be written, using either the native scripting features in a viewer, or in an external scripting language, to format a structure file in a format that is recognized by a particular viewer.

Another method according to the invention, illustrated in FIG. 5, comprises the steps of: 1) identifying one or more alignment domain in each said sequence 1; 2) selecting a master sequence comprising one or more alignment domains from the sequences 3; 3) displaying to the user: i) a first graphical user interface comprising a first multiple sequence alignment representation, a means for the user to identify a new master sequence; a means for the user to select one or more master sequence alignment domains; and a means for the user to select one or more sequences for subsequent processing of their corresponding structures; and ii) a second graphical user interface comprising a phylogenetic tree representation of each sequence that comprises the multiple sequence alignment representation 11; 4) receiving at least one master sequence alignment domain selection made by a user using the means for selecting a master sequence alignment domain 7; 5) displaying to the user via the first and second graphical user interfaces, respectively: i) a second multiple sequence alignment representation wherein the sequences that comprise the multiple sequence alignment are shifted such that the selected master sequence alignment domain(s) are aligned with their respective homologous alignment domains that comprise the other sequences in the multiple sequence alignment representation; and ii) for each selected master sequence alignment domain, a phylogenetic tree representation of the selected master sequence alignment domain and its homologous alignment domains that comprise the other sequences in the multiple sequence alignment 12; 6) receiving at least one sequence selection made by a user using said means for selecting a sequence 9; and 7) identifying the protein structures corresponding to the selected sequences for subsequent processing 13.

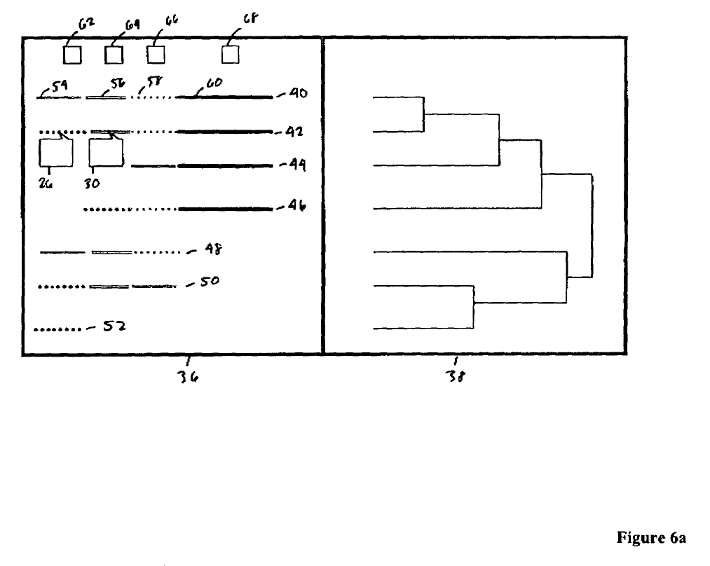

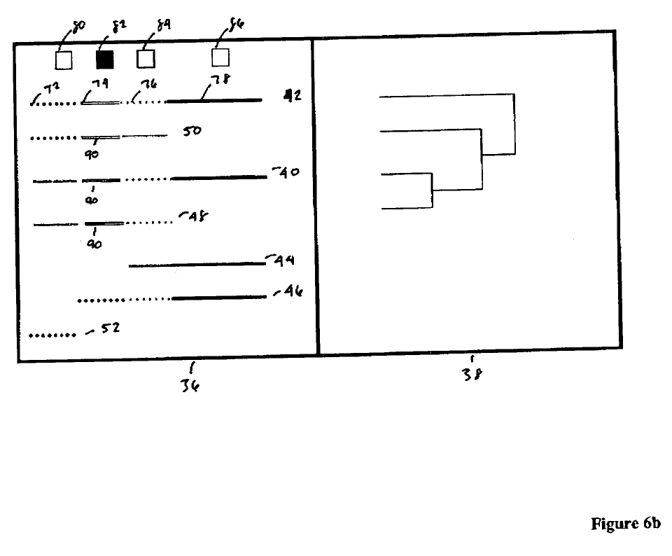

FIGS. 6a and 6b illustrate exemplary GUIs according to this aspect of the invention. FIG. 6a illustrates for the case of seven sequences 40, 42, 44, 46, 48, 50, 52 corresponding to seven structures, a first graphical user interface 36 comprising a first multiple sequence alignment representation of the sequences, a means for the user to identify a new master sequence; a means for the user to select one or more master sequence alignment domains; and a means for the user to select one or more sequences and ii) second graphical user interface 38 comprising a phylogenetic tree representation of each sequence that comprises the multiple sequence alignment representation. The uppermost sequence is the default master sequence 40. Because it comprises four master sequence alignment domains 54, 56, 58, 60, the user is also presented four corresponding check boxes 62, 64, 66, 68 as the means for selecting a master sequence alignment domain. Like in FIGS. 2-3, the means to promote a sequence to master sequence and the means to select a sequence for further processing of its corresponding structure are user activated dialog boxes 26, 30.

FIG. 6b illustrates the first and second graphical user interfaces 36, 38 illustrated in FIG. 6a after a user has selected sequence 42 as the master sequence and selected the second master sequence alignment domain. Because the new master sequence 42 comprises four master sequence alignment domains 72, 74, 76, 78, the user is again presented four corresponding check boxes 80, 82, 84, 86. A user has selected the second check box 82 corresponding to the second master sequence alignment domain. By selecting the second check box 82, the sequences are arranged vertically such that the three sequences 50, 40, 48, that comprise a domain homologous 90 to the second master sequence alignment domain 74 are grouped together and shifted horizontally about this domain 90. In the second GUI 38, the phylogenetic tree is built from the master sequence alignment domain 74 and its homologous alignment domains 90.

A phylogenetic tree comprises one or more branches connected to a root. Methods for generating phylogenetic trees are well known in the art. Chapter 14 in Baxevanis, A., Ouellette, B.F., Bioinformatics A Practical Guide to the Analysis of Genes and Proteins, (Wiley Interscience 2001) reviews in great detail the various methods that may be employed to generate a phylogenetic tree. A general method for determining a phylogenetic tree for a plurality of alignment domains (or sequences) comprises the steps of: 1) determining an alignment score matrix using a sequence-sequence alignment tool for each potential pair-wise combination of alignment domains (sequences); 2) determining an evolutionary distance matrix from the alignment score matrix; and 3) building a phylogenetic tree based upon the evolutionary distance matrix. Methods for determining sequence-sequence alignment scores are well known in the art and include the various BLAST, Smith-Waterman, FASTA and ClustalW algorithms. See Baxevanis, A., Ouellette, B. F., Bioinformatics A Practical Guide to the Analysis of Genes and Proteins, (Wiley Interscience 2001) at Ch. 8.

An evolutionary distance matrix may be formed by converting each alignment score in the alignment score matrix to a distance. As one ordinarily skilled the art appreciates, the higher an alignment score is between two sequences, the more evolutionarily similar those two sequences are. In the limit of perfect alignment, two sequences may be said to have an evolutionary distance equal to zero between them. One method used by the methods according to the invention determines an evolutionary distance between two sequences, i and j, according to d_{i,j}=1−I_{i,j} where I_{i,j}=S_{i,j}/L. I_{i,j} is referred to as the fractional identity score. S_{i,j} is the alignment score formed by summing the pairwise residue alignments along the highest scoring alignment that may be formed between i and j. L is the length of the alignment including gaps. In order to illustrate this method consider the following alignment between two exemplary sequences: NEQKRMPSRKFC and NEQKRRK. Assume the optimal alignment between these two sequences is:

NEQKRMPSRKFC
NEQKR___RK__{—}

Accordingly, S_{i,j}=7, L=12, I_{i,j}=0.583 and d_{i,j}=0.417.

A phylogenetic tree may be built from an evolutionary distance matrix using any method known in the art for tree building, including but not limited to, the Unweighted Pair Group Method, the Neighbor Joining Method, the Fitch-Margoliash Method and the Minimum Evolution Method. All these methods are detailed in Chapter 14 of Baxevanis, A., Ouellette, B. F., Bioinformatics A Practical Guide to the Analysis of Genes and Proteins (Wiley Interscience 2001) with secondary citations. They are also detailed with examples in Durbin, R., Eddy, S., Krogh, A., and Mitchison, G., Biological Sequence Analysis, (Cambridge University Press, Cambridge, U.K., 1998).

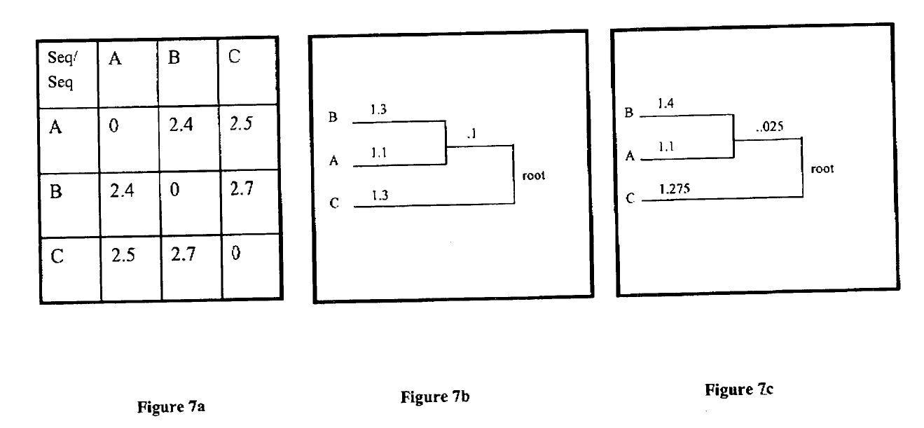

FIGS. 7a-c illustrate the relationship between an evolutionary distance matrix and its corresponding phylogenetic tree. FIG. 7a represents an exemplary evolutionary distance matrix comprising three sequences, denoted as A, B, and C. FIGS. 7b and 7c represent the two phylogenetic trees corresponding to the evolutionary distance matrix illustrated in FIG. 7a. Both phylogenetic trees were determined using the Neighbor Joining Method. In one embodiment of the invention, the phylogenetic tree represented in FIG. 7b is preferred since it minimizes the distance (1.4 distance units in FIG. 7b versus 1.425 distance units in FIG. 7c) between the root and the most distant leaf, B.

Phylogenetic trees provide important additional information to the user beyond that which can be provided from the multiple sequence alignment representation. While the multiple sequence alignment representation permits the user to quickly understand which if any sequences among a plurality of sequences comprise master sequence alignment domains, it provides no meaningful information regarding the degree of similarity of the selected domains (or sequences). By contrast, a phylogenetic tree allows the user to immediately determine the relative sequence similarity among the selected domain regions or sequences as a whole. One embodiment according to the invention simultaneously presents to the user the multiple sequence alignment representation and the phylogenetic tree representations side-by-side in a single window. When the two representations are presented to the user side-by-side, a user may very quickly determine which if any sequences comprise a relevant alignment domain(s) and the similarity of those alignment domains.

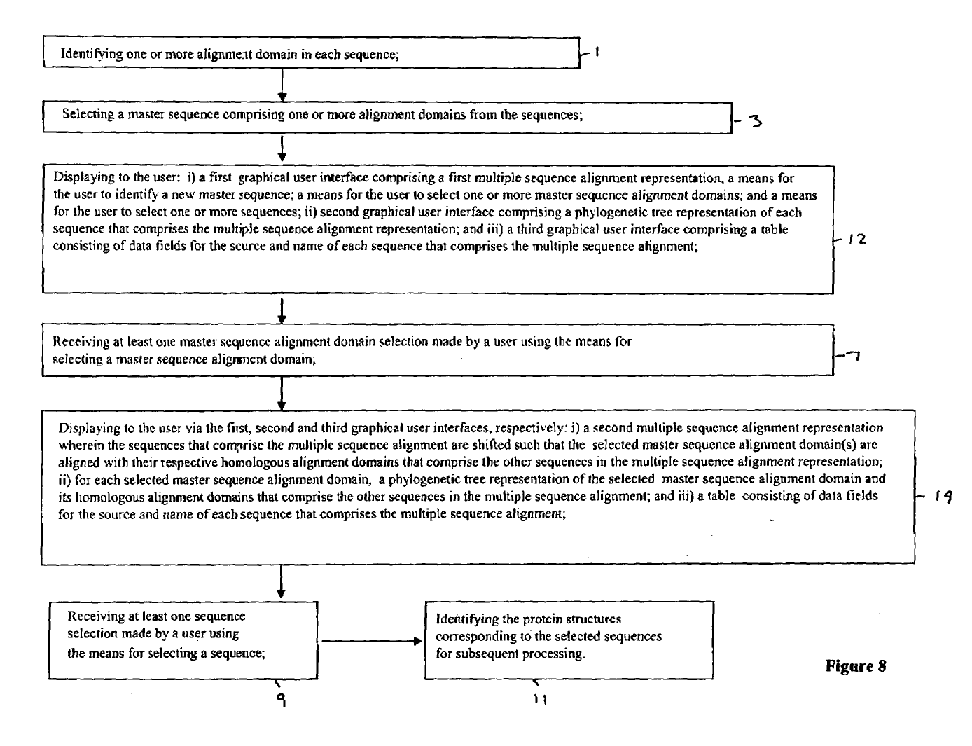

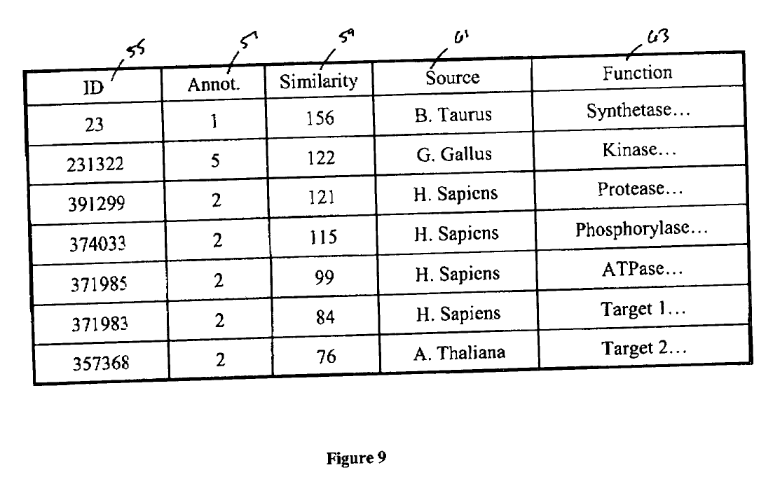

A still further method according to the invention, illustrated in FIG. 8, comprises the steps of: 1) identifying one or more alignment domain in each said sequence 1; 2) selecting a master sequence comprising one or more alignment domains from said sequences 3; 3) displaying to the user: i) a first graphical user interface comprising a first multiple sequence alignment representation; a means for the user to identify a new master sequence; a means for the user to select one or more master sequence alignment domains; and a means for the user to select one or more sequences; ii) a second graphical user interface comprising a phylogenetic tree representation of each sequence that comprises the multiple sequence alignment representation; and iii) a third graphical user interface comprising a table consisting of data fields for the source and name of each sequence that comprises the multiple sequence alignment 14; 4) receiving at least one master sequence alignment domain selection made by a user using the means for selecting a master sequence alignment domain 7; 5) displaying to the user via the first, second and third graphical user interfaces, respectively: i) a second multiple sequence alignment representation wherein the sequences that comprise the multiple sequence alignment are shifted such that the selected master sequence alignment domain(s) are aligned with their respective homologous alignment domains that comprise the other sequences in the multiple sequence alignment representation; ii) for each selected master sequence alignment domain, a phylogenetic tree representation of the selected master sequence alignment domain and its homologous alignment domains that comprise the other sequences in the multiple sequence alignment; and iii) a table consisting of data fields for the source and name of each corresponding sequence that comprises the multiple sequence alignment;16; 6) receiving at least one sequence selection made by a user using said means for selecting a sequence 9; and 7) identifying the protein structures corresponding to the selected sequences for subsequent processing 13.

Further optional data fields include: 1) a score indicating the relative amount of information known relating to a particular sequence, for example, the level of functional and/or structural annotation of the sequence or its corresponding structure; 2) a sequence similarity score, such as the fractional identity score, that reflects the similarity of a particular sequence to the master sequence or the similarity of a master sequence alignment domain to a homologous domain; and 3) a database or sequence identification number that cross-references a sequence to a database schema. Functional and/or structural annotation of a sequence and/or its corresponding structure refers to the further identification of such structural and functional aspects of proteins, such as sequence domains, structure domains, biological function or the identification of small molecule binding sites. In one embodiment of the invention those data fields corresponding to sequences comprising alignment domains selected by the user in step 4) are highlighted relative to those tabular entries corresponding to sequences which do not comprise a selected alignment region(s).

FIG. 9 shows an exemplary table representation, for seven hypothetical sequences, comprising data fields for: 1) a sequence identification number 55; 2) an annotation score 57; 3) a relative sequence similarity score 59; 4) the source of the sequence 61; and 5) a functional identification of the sequence 63. As one ordinarily skilled in the art will recognize, there is no inherent limitation on the nature of the tabular information relating to a sequence that may be provided. Accordingly, any table representation comprising other sequence related data in combination with a multiple sequence alignment representation and a phylogenetic tree representation is within the ambit of the present invention.

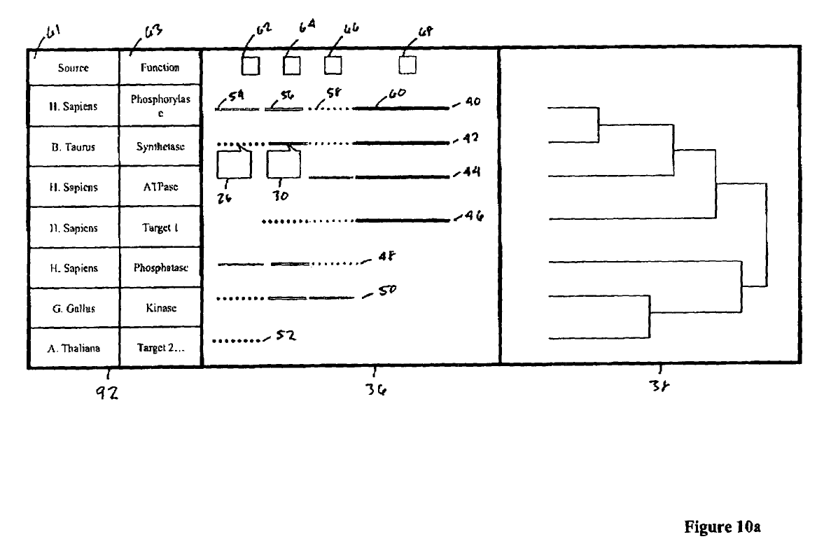

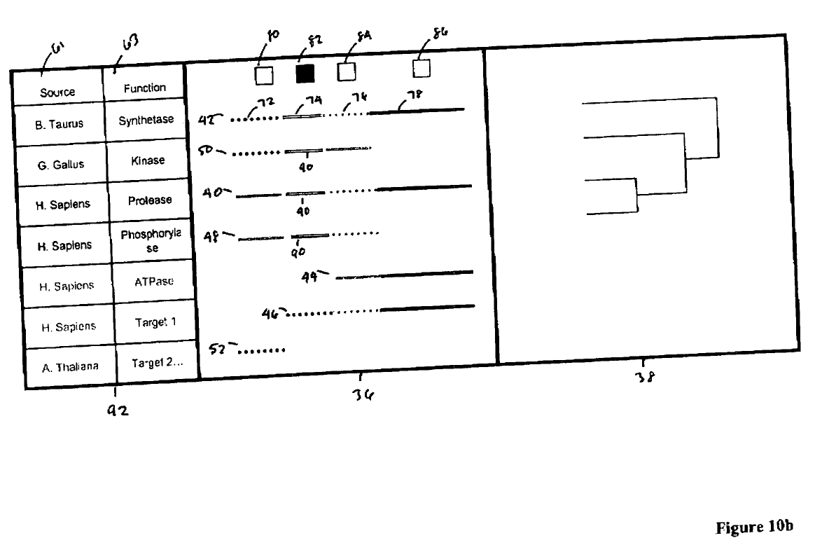

FIGS. 10a and 10b show exemplary GUIs according to this aspect of the invention. FIGS. 10a and 10b corresponds identically to FIGS. 5a and 5b with the exception of a third GUI 92 comprising a table with data fields for the source and name of the corresponding sequences that comprise the multiple sequence alignment. In one embodiment of the invention, illustrated in FIG. 10b, the data fields corresponding to sequences 42, 50, 40, 48 are highlighted because they comprise either the selected alignment domain 82 or its homologous alignment domains 90. In the right pane, only those sequences 81, 83, 85, 87 which comprise the master sequence alignment domain 97 or its homologous alignment domains 111 participate in the phylogenetic tree representation. By presenting the user a table with sequence function and source information in combination with the multiple sequence representation and the phylogenetic tree representations the user is presented a continuum of similarity information. At the broadest similarity level, the table of sequence function and source informs the user of the function and source of a particular structure. At a more detailed level, the multiple sequence representation informs the user of which sequences comprise master sequence alignment domains. And at a still more detailed level, the phylogenetic tree informs the user of the relative similarity of selected alignment domains.

Systems According to the Invention

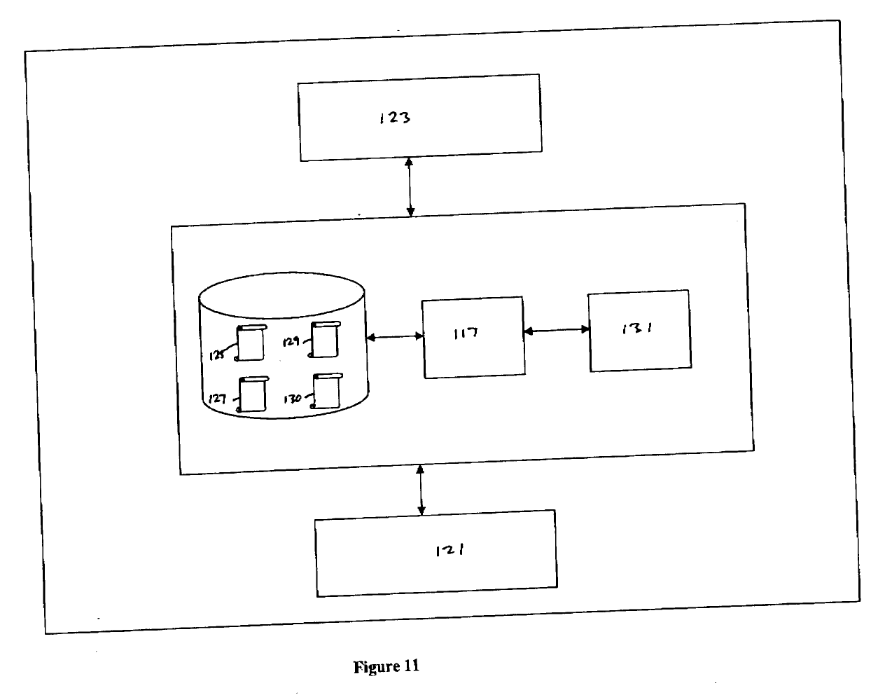

In general, as is shown in FIG. 11, a system according to the invention 115 comprises a processor 117, a memory 119, optionally, an input device 121, optionally, an output device 123, programming for an operating system 125, programming for the methods according to the invention 127, optionally, programming for storing and retrieving a plurality of sequences and structures 129, optionally, programming for displaying protein structures based upon their structural coordinates 130. The systems according to the invention may, optionally, also comprise a device for networking to another device 131.

A processor 117, as used herein, may include one or more microprocessor(s), field programmable logic array(s), or one or more application specific integrated circuit(s). Exemplary processors include, but are not limited to, Intel Corp.'s Pentium series processor (Santa Clara, Calif.), Motorola Corp.'s PowerPC processors (Schaumberg, Ill.), MIPS Technologies, Inc.'s MIPs processors (Mountain View, Calif.), or Xilinx, Inc.'s Vertex series of field programmable logic arrays (San Jose, Calif.).

A memory 119, as used herein, is any electronic, magnetic or optical based media for storing, reading and writing digital information or a combination of such media. Exemplary types of memory include, but are not limited to, random access memory, electronically programmable read-only memory, flash memory, magnetic based disk and tape drives, and optical based disk drives. The memory stores: 1) programming for the methods according to the invention; 2) programming for an operating system and 3) programming for storing and retrieving a plurality of protein sequences and structures.

An input device 121, as used herein, is any device that accepts and processes information from a user. Exemplary devices include, but are not limited to, a keyboard and a pointing device such as a mouse, trackball, joystick or a touch screen/tablet, a microphone with corresponding speech recognition software, any removable, optical, magnetic or electronic media based drive, such as a floppy disk drive, a removable hard disk drive, a Compact Disk/Digital Video Disk drive, a flash memory reader or some combination thereof.

An output device 123, as used herein, is any device that processes and outputs information to a user. Exemplary devices include, but are not limited to, visual displays, speakers and or printers. A visual display may be based upon any technology known in the art for processing and presenting a visual image to a user, including, cathode ray tube based monitors/projectors, plasma based monitors, liquid crystal display based monitors, digital micro-mirror device based projectors, or light-valve based projectors.

Programming for an operating system 125, as used herein, refers to any machine code, executed by the processor, 117, for controlling and managing the data flow between the processor 117, the memory, the input device 121, the output device 123, and any networking devices 131. In addition to managing data flow among the hardware components that comprise a computer system, an operating system also provides, scheduling, input-output control, file and data management, memory management, and communication control and related services, all in accordance with known methodologies. Exemplary operating systems include, but are not limited to, Microsoft Corp's Windows and NT (Redmond, Wash.), Sun Microsystem, Inc.'s Solaris Operating System (Palo Alto, Calif.), Red Hat Corp.'s version of Linux (Durham, N.C.) and Palm Corp.'s PALM OS (Milpitas, Calif.).

Programming for storing and retrieving a plurality of protein structures and sequences 129, as used herein, refers to machine code, that when executed by the processor, allows for the storing, retrieving and organizing of protein structures and sequences. Exemplary software includes, but is not limited to, relational and object oriented databases such as Oracle Corp.'s 9i (Redwood City, Calif.), International Business Machine, Inc.'s DB2 (Armonk, N.Y.), Microsoft Corp.'s Access (Redmond, Wash.) and Versant Corp.'s (Freemont, Calif.) Versant Developer Suite 6.0. If protein sequences and structures are stored as flat files, programming for storing and retrieving a plurality of structures and sequences includes programming for operating systems.

Programming for the methods according to the invention 127, as used herein, refers to machine code, that when executed by the processor, performs the methods according to the invention.

Programming for displaying protein structures based upon their structural coordinates 130, as used herein, refers to machine code, that when executed by the processor, displays protein structures to the user via the output device, 123, based upon their structural coordinates. Exemplary software for displaying protein structures includes but is not limited to, Rasmol, available for download at http://www.rasmol.org/, Cn3D available for download at http://www.ncbi.nlm.nih.gov/Structure/CN3D/cn3d.shtml, Molscript, available for download at, http://www.avatar.se/molscript/, MolMol available for download at http://www.mol.biol.ethz.ch/wuthrich/software/molmol/, and the Insight II software suite available from Accelrys, Inc., (San Diego, Calif.).

A networking device 131 as used herein refers to a device that comprises the hardware and software to allow a system according to the invention to electronically communicate either directly or indirectly to a network server, network switch/router, personal computer, terminal, or another communications device over a distributed communications network. Exemplary networking schemes may be based on packet over any media and include, but are not limited to, Ethernet 10/100/1000, IEEE 802.11x, SONET, ATM, IP, MPLS, IEEE 1394, xDSL, Bluetooth, or any other ANSI approved standard.

It will be appreciated by one skilled in the art that the programming for an operating system 125, the programming for storing and retrieving a plurality of protein structures and sequences 129, and the programming for the methods according to the invention 127 may be loaded onto a system according to the invention through either the input device 121, a networking device 131 or a combination of both.

Systems according to the invention may be based upon personal computers (“PCs”) or network servers programmed to perform the methods according to the invention. A suitable server and hardware configuration is an enterprise class Pentium based server, comprising an operating system such as Microsoft Corp.'s NT, Sun Microsystems, Inc.'s Solaris or Red Hat Corp.'s version of Linux with 1 GB random access memory, 100 GB storage, either a line area network communications card, such as a 10/100 Ethernet card or a high speed Internet connection, such as a T1/E1 line, optionally, an enterprise database and programming for the methods according to the invention. The storage and memory requirements listed above are not intended to represent minimum hardware configurations, rather they represent a typical server system which may be readily purchased from vendors at the time of filing. Such servers may be readily purchased from Dell, Inc. (Austin, Tex.), or Hewlett-Packard, Inc. (Palo Alto, Calif.) with all the features except for the enterprise database, programming for displaying protein structures based upon their structural coordinates and the programming for the methods according to the invention. Enterprise class databases may be purchased from Oracle Corp. or International Business Machines, Inc. It will be appreciated by one skilled in the art that one or more servers may be networked together. Accordingly, the programming for the methods according the invention and an enterprise database for storing and retrieving a plurality of protein structures and sequences may be stored on physically separate servers in communication with one another.

A suitable desktop PC and hardware configuration is a Pentium based desktop computer comprising at least 128 MB of random access memory, 10 GB of storage, a Windows or Linux based operating system, optionally, either a line area network communications card, such as a 10/100 Ethernet card or a high speed Internet connection, such as a T1/E1 line, optionally, a TCP/IP web browser, such as Microsoft Corp.'s Internet Explorer or the Mozilla Web Browser, optionally, a database such as Microsoft Corp.'s Access, programming for displaying protein structures based upon their structural coordinates and programming for the methods according to the invention. Once again, the exemplary storage and memory requirement are only intended to represent PC configurations which are readily available from vendors at the time of filing. They are not intended to represent minimum configurations. Such PCs may be readily purchased from Dell, Inc. or Hewlett-Packard, Inc. (Palo Alto, Calif.) with all the features except for the programming for the methods according to the invention.

Although the invention has been described with reference to preferred embodiments and specific examples, it will be readily appreciated by those skilled in the art that many modifications and adaptations of the invention are possible without deviating from the spirit and scope of the invention. Thus, it is to be clearly understood that this description is made only by way of example and not as a limitation on the scope of the invention. All references herein are hereby incorporated by reference.

What is claimed is:

1. A method for sorting a plurality of protein structures and corresponding sequences wherein both the protein structures and sequences are generated from a database query comprising the steps of:
a. identifying one or more alignment domains in each said sequence;
b. selecting a master sequence comprising one or more alignment domains from said sequences;
c. displaying to the user a graphical user interface comprising:
i. a first multiple sequence alignment representation comprising: a multiple sequence alignment between said master sequence and said other sequences; identifications of at least one master sequence alignment domain; and identifications of at least at least one alignment domain in each said other sequence that comprises said multiple sequence aligmnent;
ii. a means for the user to identify a new master sequence;
iii. a means for the user to select one or more master sequence alignment domains; and
iv. a means for the user to select to one or more sequences in order to select their corresponding structures for processing.
d. receiving at least one master sequence alignment domain selection made by a user using said means for selecting a master sequence alignment domain;
e. displaying to the user a second multiple sequence alignment representation wherein the sequences that comprise the multiple sequence alignment are shifted such that said selected master sequence alignment domain(s) are aligned with their respective homologous alignment domains that comprise the other said sequences in said multiple sequence alignment;
f. receiving at least one sequence selection made by a user using said means for selecting a sequence in order to select its corresponding structure; and
g. identifying the protein structures corresponding to the selected sequences thereby sorting the protein structure into those selected by the user for subsequent processing and those structures that are not selected by the user for subsequent processing.

2. The method of claim 1 wherein said alignment domains correspond to domain annotations found in the Pfam database, SMART or the COG database.

3. The methods of claim 1 wherein said subsequent processing is the visualization of said protein.

4. A method for sorting a plurality of protein structures and corresponding sequences wherein both the protein structures and sequences are generated from a database query comprising the steps of:
a. identifying one or more alignment domains in each said sequence;
b. selecting a master sequence comprising one or more alignment domains from said sequences;
c. displaying to the user, a first graphical user interface comprising:
i. a first multiple sequence alignment representation comprising: a multiple sequence alignment between said master sequence and said other sequences; identifications of at least one master sequence alignment domain; and identifications of at least at least one alignment domain in each said other sequence that comprises said multiple sequence alignment;
ii. a means for the user to identify a new master sequence;
iii. a means for the user to select one or more master sequence alignment domains; and
iv. a means for the user to select to one or more sequences in order to select their corresponding structures for processing; and
a second graphical user interface comprising a phylogenetic tree representation of each sequence that comprises said multiple sequence alignment representation;
d. receiving at least one master sequence alignment domain selection made by a user using said means for selecting a master sequence alignment domain;
e. displaying to the user via said first and second graphical user interfaces, respectively:
i. a second multiple sequence alignment representation wherein the sequences that comprise the multiple sequence alignment are shifted such that said selected master sequence alignment domain(s) are aligned with their respective homologous alignment domains that comprise the other said sequences in said multiple sequence alignment; and
ii. for each selected master sequence alignment domain, a phylogenetic tree representation of the selected master sequence alignment domain and its homologous alignment domains that comprise the other said sequences in said multiple sequence alignment;
f. receiving at least one sequence selection made by a user using said means for selecting a sequence in order to selects its corresponding structure for processing; and
g. identifying the protein structures corresponding to the selected sequences thereby sorting the protein structure into those selected by the user for subsequent processing and those structures that are not selected by the user for subsequent processing.

5. The method of claim 4 wherein said alignment domains correspond to domain annotations found in the Pfam database, SMART database or the COG database.

6. The methods of claim 4 wherein said subsequent processing is the visualization of said protein.

7. A method for sorting a plurality of protein structures and corresponding sequences wherein both the protein structures and sequences are generated from a database query comprising the steps of:
a. identifying one or more alignment domain in each said sequence;
b. selecting a master sequence comprising one or more alignment domains from said sequences;
c. displaying to the user, a first graphical user interface comprising:
i. a first multiple sequence alignment representation comprising: a multiple sequence alignment between said master sequence and said other sequences; identifications of at least one master sequence alignment domain; and identifications of at least at least one alignment domain in each said other sequence that comprises said multiple sequence alignment;
ii. a means for the user to identify a new master sequence;
iii. a means for the user to select one or more master sequence alignment domains; and
iv. a means for the user to select to one or more sequences in order to select their corresponding structures for processing;
a second graphical user interface comprising a phylogenetic tree representation of each sequence that comprises the multiple sequence alignment representation; and
a third graphical user interface comprising a table consisting of data fields for the source and name of each corresponding sequence that comprises the multiple sequence alignment;
d. receiving at least one master sequence alignment domain selection made by a user using said means for selecting a master sequence alignment domain;
e. displaying to the user via said first, second and third graphical user interfaces, respectively:
i. a second multiple sequence alignment representation wherein the sequences that comprise the multiple sequence alignment are shifted such that said selected master sequence alignment domain(s) are aligned with their respective homologous alignment domains that comprise the other said sequences in said multiple sequence alignment;
ii. for each selected master sequence alignment domain, a phylogenetic tree representation of the selected master sequence alignment domain and its homologous alignment domains that comprise the other said sequences in said multiple sequence alignment; and
iii. a table consisting of data fields for the source and name of each corresponding sequence that comprises the multiple sequence alignment;
f. receiving at least one sequence selection made by a user using said means for selecting a sequence in order to select its corresponding structure for processing; and
g. identifying the protein structures corresponding to the selected sequences thereby sorting the protein structure into those selected by the user for subsequent processing and those structures that are not selected by the user for subsequent processing.

8. The method of claim 7 wherein said alignment domains correspond to domain annotations found in the Pfam database, SMART database or the COG database.

9. The methods of claim 7 wherein said subsequent processing is the visualization of said protein.

10. The method of claim 10 wherein said table further comprises data fields for:
a. a database annotation score that reflects the relative amount of information known about said sequence;
b. a sequence similarity score that reflects the evolutionary similarity of said sequence with said master sequence; and
c. a sequence identification number that identifies said sequence from a plurality of sequences in a database.

11. A computer system comprising:
a. a processor;
b. an input means;
c. an output means;
d. a memory
e. an operating system means;
f. programming for displaying to the user a graphical user interface comprising:
i. a multiple sequence alignment representation comprising: a multiple sequence alignment between a master sequence and a plurality of other sequences; identifications of at least one master sequence alignment domain; and identifications of at least at least one alignment domain in each said other sequence that comprises said multiple sequence alignment;
ii. a means for the user to identify a new master sequence;
iii. a means for the user to select one or more master sequence alignment domains; and
iv. a means for the user to select to one or more sequences in order to select their corresponding structures for processing.

12. The system of claim 11 wherein said means for the user to select one or more master sequence alignment domains comprises displaying to the user a selectable check box corresponding to each master sequence alignment domain.

13. A computer system comprising:
a. a processor;
b. an input means;
c. an output means;
d. a memory
e. an operating system means;
f. programming for displaying to the user simultaneously, a first graphical user interface comprising
i. a multiple sequence alignment representation comprising: a multiple sequence alignment between a master sequence and a plurality of other sequences; identifications of at least one master sequence alignment domain; and identifications of at least at least one alignment domain in each said other sequence that comprises said multiple sequence alignment;
ii. a means for the user to identify a new master sequence;
iii. a means for the user to select one or more master sequence alignment domains; and
iv. a means for the user to select to one or more sequences in order to select their corresponding structures for processing; and
a second graphical user interface comprising, for each selected master sequenced alignment domain, a phylogenetic tree representation between a master sequence alignment domain and a plurality of alignment domains homologous to said master sequence alignment domain.

14. The system of claim 13 wherein said means for the user to select one or more master sequence alignment domains comprises displaying to the user a selectable check box corresponding to each master sequence alignment domain.

15. A computer system comprising:
a. a processor;
b. an input means;
c. a memory;
d. an output means;
e. an operating system means;
f. programming for displaying to the user simultaneously, a first graphical user interface comprising:
i. a multiple sequence alignment representation comprising: a multiple sequence alignment between a master sequence and a plurality of other sequences; identifications of at least one master sequence alignment domain; and identifications of at least at least one alignment domain in each said other sequence that comprises said multiple sequence alignment;
ii. a means for the user to identify a new master sequence;
iii. a means for the user to select one or more master sequence alignment domains; and
iv. a means for the user to select to one or more sequences in order to select their corresponding structures for processing;
a second graphical user interface comprising, for each selected master sequenced alignment domain, a phylogenetic tree representation between a master sequence alignment domain and a plurality of alignment domains homologous to said master sequence alignment domain; and
a third graphical user interface comprising a table consisting of data fields for the source and name of each corresponding sequence that comprises the multiple sequence alignment.

16. The system of claim 15 wherein said means for the user to select one or more master sequence alignment domains comprises displaying to the user a selectable check box corresponding to each master sequence alignment domain.

17. A computer system comprising:
a. a processor;
b. a memory;
c. an input means;
d. an output means;
e. an operating system means;
f. programming for the methods according to claim 3; and
g. programming for displaying protein structures based upon their structural coordinates

18. A computer system comprising:
b. a processor;
c. a memory;
d. an input means;
e. an output means;
f. an operating system means;
g. programming for the methods according to claim 6; and
h. programming for displaying protein structures based upon their structural coordinates

19. A computer system comprising:
a. a processor;
b. a memory;
c. an input means;
d. an output means;
e. an operating system means;
f. programming for the methods according to claim 9; and
g. programming for displaying protein structures based upon their structural coordinates.
